(19) United States
(12) Patent Application Publication
(10) Pub. No.: US 2021/0393506 A1
(43) Pub. Date: Dec. 23, 2021

(54) FRAGRANCE BLENDS AND METHODS FOR PREPARATION THEREOF

(71) Applicants: Noor Al-RIFAI, Antwerpen (BE); Wundriari HOFFMANN, Camberley, Surrey (GB); Sylvain SOLINAS, Geneva (CH)

(72) Inventors: Noor Al-Rifai, Antwerpen (BE); Wundriari Hoffmann, Camberley, Surrey (GB); Sylvain Solinas, Geneva (CH)

(21) Appl. No.: 17/288,440

(22) PCT Filed: Nov. 6, 2019
(86) PCT No.: PCT/US2019/060036

§ 371 Date: Apr. 23, 2021

Related U.S. Application Data
(60) Provisional application No. 62/756,321, filed on Nov. 6, 2018.

(51) Int. Cl. A61K 8/92; A61Q 13/00

(52) U.S. Cl. CPC A61K 8/922; A61Q 13/00

(57) ABSTRACT

Provided is a method for providing a fragrance blend customized for an individual. Also provided is a method of enhancing a scent in a fragrance. Also provided are fragrance blends customized for the individual or having an enhanced scent.

### CLAIM OF PRIORITY

This patent application claims the benefit of priority to U.S. Provisional Application Ser. No. 62/756,321, filed Nov. 6, 2018, which is incorporated by reference herein in its entirety.

Perfumes with different fragrances have been used for thousands of years. The industrial production of essential oils and the synthesis of odorous substances improved both their availability and range of application. Perfumery focuses on the creation of new fragrances, which sometimes are composed of more than 100 individual components, and on fixing the base note in order to increase clinging and adhesion to the user's skin. Since perfumes have become a mass product, users wish to have different fragrances at their disposal and to be able to rapidly change the fragrance in some cases. Up to now, the only possibility for the user to do so consists in taking along several different perfumes and using them according to his/her wishes.

There is a significant unmet need for new fragrances that can be customized by the individual to reflect the individual's personal preferences. The present disclosure fulfills these and other needs, as evident in reference to the following disclosure.

Provided herein is a method for providing a fragrance blend customized for an individual, the method comprising:

providing a fragrance wherein the fragrance comprises perfuming ingredients belonging to all levels of a fragrance pyramid;

presenting the individual with a scent additive choice comprised of a plurality of scent additives;

receiving from the individual a selection of at least one scent additive from said scent additive choice, and

adding to the fragrance the selected at least one scent additive to yield a fragrance blend customized for the individual,

wherein the fragrance has a first fragrance, the selected scent additive has a second fragrance, and the fragrance blend has a third fragrance.

Also provided is a method of enhancing a scent in a fragrance, the method comprising:

providing a fragrance wherein the fragrance comprises perfuming ingredients belonging to all levels of a fragrance pyramid;

presenting the individual with a scent additive choice comprised of a plurality of scent additives wherein the plurality of scent additives have been selected to enhance an olfactive note in the fragrance;

receiving from the individual a selection of at least one scent additive from said scent additive choice, and

adding to the fragrance the selected at least one scent additive in an amount sufficient to enhance an olfactive note in the fragrance to yield a fragrance blend having an enhanced scent,

wherein the fragrance has a first fragrance, the selected scent additive has a second fragrance, and the fragrance blend has a third fragrance.

Also provided is a fragrance blend comprising a fragrance and a scent additive. Also provided is a fragrance blend comprising a fragrance, a scent additive, and an effect additive.

Also provided is a fragrance blend prepared by any of the methods described herein.

These and other objects of the invention are described in the following paragraphs. These objects should not be deemed to narrow the scope of the invention.

### BRIEF DESCRIPTION OF THE FIGURES

FIG. 1a is a diagram of the classical fragrance pyramid structure of the prior art. FIG. 1b is a diagram of a fragrance diamond construction.

FIG. 2 shows a fragrance wheel showing the inferred relationships among olfactory groups based upon similarities and differences in their odor.

FIG. 3 shows an automated perfume dispenser which can be used to perform the methods described herein.

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “one embodiment” or “an embodiment” or “some embodiments” or “a certain embodiment” means that a particular feature, structure or characteristic described in connection with the embodiment is included in at least one embodiment. Thus, the appearances of the phrases “in one embodiment” or “in an embodiment” or “in some embodiments” or “in a certain embodiment” in various places throughout this specification are not necessarily all referring to the same embodiment. Furthermore, the particular features, structures, or characteristics may be combined in any suitable manner in one or more embodiments.

Also, as used in this specification and the appended claims, the singular forms “a,” “an,” and “the” include plural referents unless the content clearly dictates otherwise.

As used herein, “a fragrance construct” refers to a concept used to visualize a fragrance's life-cycle and includes, e.g., a fragrance pyramid (including a classical fragrance pyramid, a bottom-heavy fragrance pyramid, and a top-heavy fragrance pyramid) or a fragrance diamond.

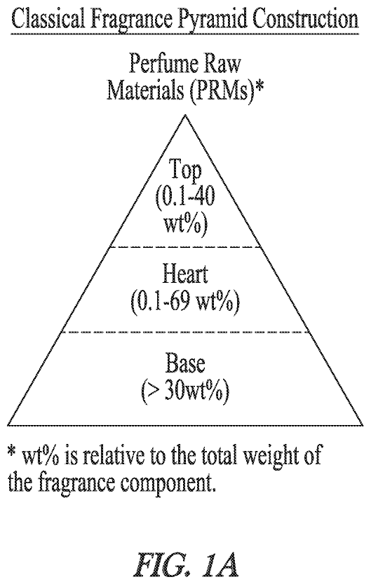

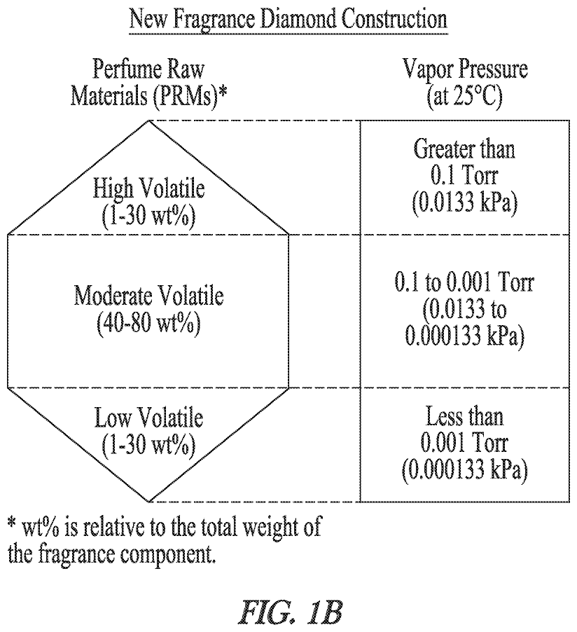

As used herein, “a fragrance pyramid” classically has three levels, as shown in FIG. 1a. The three levels may comprise a “top” level comprising “top” notes that appear first upon application. The top notes may e.g. comprise the fruity note and the aromatic note. The three levels may further comprise a “middle” level comprising “middle” notes that are the “heart” of a fragrance blend and that appear when the top notes fade. The middle notes may e.g. comprise the floral note, the green note, the fruity note, and a spicy note. The three levels may further comprise a “base” level comprising “base” notes that appear last but stay up to 24 hours. The top notes may e.g. comprise the woody notes, and a balsam note. See, e.g., Tables 4 to 10 of WO 2018/002298 which describe top, middle and bottom note perfuming compounds for watery, green, fruity, citrus, sweet, gourmand and woody notes respectively. In some embodiments, perfuming compounds having a volatility ranging from 70,000 to 100 μg/l in air are referred to as “top notes” or “high volatility”. In some embodiments, perfuming compounds having a volatility ranging from 99 to 50 μg/l in air are referred to as “middle notes” or “moderate volatility”. In some embodiments, perfuming compounds having a volatility ranging from 49 to 0.1 μg/l in air are referred to as “bottom notes” or “low volatility”. With the classical fragrance pyramid structure, base notes make up from greater than 30 wt %, typically greater than 40 wt % or typically greater than 50 wt %, relative to the total weight of the perfume formulation. The middle notes make up from about 0.1 wt % to about 60 wt % relative to the total weight of the perfume formulation and have an intermediate volatility. The top notes make up from about 0.1 wt % to about 40 wt % relative to the total weight of the perfume formulation.

In some embodiments, the fragrance pyramid comprises a top-heavy scent pyramid wherein the low volatility perfuming compounds are present in an amount of from about 1 wt % to about 30 wt % relative to the total weight of the fragrance; the moderate volatility perfuming compounds are present in an amount of from about 25 wt % to about 65 wt % relative to the total weight of the fragrance, and the high volatility perfuming compounds are present in an amount of greater than about 30 wt % relative to the total weight of the fragrance. See, PCT application number PCT/US18/43700.

In some embodiments, the fragrance pyramid comprises a bottom-heavy scent pyramid wherein the low volatility perfuming compounds are present in an amount of greater than about 30 wt % relative to the total weight of the fragrance; the moderate volatility perfuming compounds are present in an amount of from about 30 wt % to about 70 wt % relative to the total weight of the fragrance, and the high volatility perfuming compounds are present in an amount of from about 0.1 wt % to about 30 wt % relative to the total weight of the fragrance. See, PCT application number PCT/US18/43721.

As used herein, “a fragrance diamond” means a fragrance formulation as shown in FIG. 1b. In particular, the diamond construction relates to the relative weight % of the fragrance materials classified according to their vapor pressure category (i.e., low, moderate or high). A diamond constructed fragrance has a substantially greater amount of the perfume raw materials of a moderate volatility as compared to the low and high volatile fragrance materials. See, e.g., US Patent Publication No. 2016-0362630.

As used herein, “accord” refers to the basic character of a fragrance composition.

As used herein, the term “odor descriptor” or “olfactive note” or “note” refers to a description of the odor character of a fragrance or a perfuming compound. Several procedures for describing odor characters are known. See, e.g, M. Zarzo and David Stanton in Attention, Perception and Psychophysicy, 2009, 71(2) 225-247.

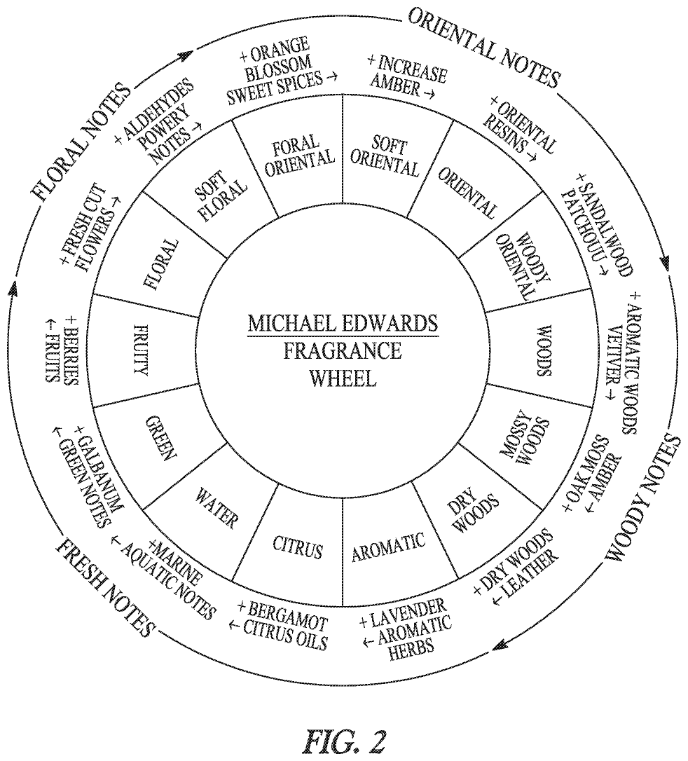

As used herein, a “scent family” is a group of olfactive notes of a fragrance wheel that give a similar or common olfactory impression, e.g. a “fresh”, “floral”, “oriental”, or “woody”. The fragrance wheel may be a fragrance wheel according to Michael Edwards. See, e.g., FIG. 2. For example, the floral family may be divided into the notes “floral” and “soft floral”. The fresh family may be divided into the notes “aromatic”, “citrus fruity”, “green”, “fruity”, and “water”. The oriental family may be divided into the notes “floral oriental”, “soft oriental”, “oriental”, and “woody oriental”. The woody family may be divided into the notes “woods”, “mossy woods”, and “dry woods”.

As used herein, the term “contrasting notes” is meant to designate olfactive notes belonging to scent families that are distant from one another. Olfactive notes adjacent to each other on the fragrance wheel are not considered contrasting olfactive notes. However, olfactive notes separated by at least one adjacent olfactive note are considered contrasting olfactive notes, wherein the degree of contrast is directly proportional to the degree of separation.

As used herein, the term “perfuming ingredient” or “perfuming compound” refers to a compound having the primary purpose of conferring or modulating an odor. The skilled person is able to select the nature and type of the perfuming ingredients on the basis of its general knowledge and according to intended use or application and the desired effect. In general terms, perfuming ingredients belong to chemical classes as varied as alcohols, aldehydes, ketones, esters, ethers, acetates, nitriles, terpenoids, nitrogenous or sulphurous heterocyclic compounds and essential oils, and said perfuming co-ingredients can be of natural or synthetic origin. See, e.g., S. Arctander, Perfume and Flavor Chemicals, 1969, Montclair, N.J., USA, or its more recent versions, or in other works of a similar nature. See also, U.S. Pat. No. 4,534,891 and Perfumes, Cosmetics and Soaps, Second Edition, edited by W. A. Poucher, 1959, or its more recent versions.

As used herein, “classic” refers to a fragrance that has been widely accepted and enjoyed popularity for a minimum of 10 years.

As used herein, “commercial” refers to a fragrance that currently is or previously has been available for purchase by the general public.

As used herein, the terms “modulator” and “fixative” are used interchangeably to designate an agent having the capacity to affect the fragrance profile, such as, for example, by impacting the fragrance materials' evaporation rate. The modulator may mediate its effect by lowering the vapor pressure of the fragrance materials and increasing their adherence to the substrate (skin and/or hair) thus ensuring a longer-lasting impression of the fragrance. By incorporating the modulator, it is desired that the fragrance profile, like the volatile fragrance materials components, of the composition can be perceived by an observer or user thereof, over a longer period of time, as compared to the same perception in the absence of the modulator. In certain embodiments, the modulators are used with a reduced level of low volatile fragrance materials (i.e., a fragrance material having a vapor pressure <0.001 Torr at 25° C.)

As used herein, the term “co-modulator” means an agent that is added to the composition in addition to the modulators and has the similar function of impacting the evaporation rate and intensity of the fragrance materials, so as to improve or prolong the perception of the fragrance profile by the consumer. Examples of co-modulators include, without limitation, isocetyl alcohol (e.g., CERAPHYL ICA; see PCT Publication No. WO2013/64412 (Firmenich)), PPG-3 myristyl ether (e.g., Tegosoft APM and/or Varonic APM), Neopentyl glycol diethylhexanoate (e.g., Schercemol NGDO), and mixtures thereof.

As used herein, the term “non-odorous” means an agent that does not impart an odor of its own when added into an exemplary composition. For example, a “non-odorous fragrance modulator” does not impart a new odor that alters the character of the fragrance profile of the composition to which it is added. The term “non-odorous” also encompasses an agent that may impart a minimal or slight odor of its own when added into a composition of the present invention. However, the odor imparted by the “non-odorous fragrance modulator” is generally undetectable or tends to not substantively alter the character of the fragrance profile of the composition to which it is added initially or preferably over time. Furthermore, the term “non-odorous” also includes materials that are perceivable only by a minority of people or those materials deemed “anosmic” to the majority of people. Furthermore, the term “non-odorous” also includes materials that may, from particular suppliers, contain an odor due to impurities, such as when the materials contain the impurities at not more than about 5 wt %, preferably not more than 1 wt %, often even not more than 1 part per million (ppm). These impurities maybe removed by purification techniques known in the art as required to make them suitable for use in fragrance compositions described herein. For example, a “non-odorous modulator” such as PPG-20 Methyl Glucose Ether does not impart a new odor that alters the character of the composition to which it is added.

As used herein, the term “habituating” or “habituation” refers an individual or group who has decreased sensitivity to perceiving a fragrance or fragrance material. A fragrance or fragrance material is considered habituated when their Degree of Habituation (percent change in odor detection threshold or “ODT”) is greater than 150%, greater than 300%, greater than 500%, greater than 1000% according to the method described in the Test Methods section of this specification

As used herein, the term “Odor Detection Threshold” refers to the lowest vapor concentration of that material which can be olfactorily detected. The ODT is measured according to methods known in the art. See, e.g., US Patent Publication No. 2015/0322387.

As used herein, the term “vapor pressure” means the partial pressure in air at a defined temperature for a given chemical species. It defines a chemical species' desire to be in the gas phase rather than the liquid or solid state. The higher the vapor pressure the greater the proportion of the material that will, at equilibrium, be found in a closed headspace. It is also related to the rate of evaporation of a fragrance material which is defined in an open environment where material is leaving the system. The vapor pressure is determined according to the reference program Advanced Chemistry Development (ACD/Labs) Software Version 11.02, (1994-2013).

All percentages and ratios are calculated by weight unless otherwise indicated. All percentages and ratios are calculated based on the total composition unless otherwise indicated. As used herein, “about” means±20% of the stated value, and includes more specifically values of ±10%, ±5%, ±2% and ±1% of the stated value.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Provided is a method for providing a fragrance blend customized for an individual, the method comprising:

providing a fragrance wherein the fragrance comprises perfuming ingredients belonging to all levels of a fragrance construct;

presenting the individual with a scent additive choice comprised of a plurality of scent additives;

receiving from the individual a selection of at least one scent additive from said scent additive choice, and

adding to the fragrance the selected at least one scent additive to yield a fragrance blend customized for the individual,

wherein the fragrance has a first fragrance, the selected scent additive has a second fragrance, and the fragrance blend has a third fragrance.

Also provided is a method of enhancing a scent in a fragrance, the method comprising:

providing a fragrance wherein the fragrance comprises perfuming ingredients belonging to all levels of a fragrance construct;

presenting the individual with a scent additive choice comprised of a plurality of scent additives wherein the plurality of scent additives have been selected to enhance an olfactive note in the fragrance;

receiving from the individual a selection of at least one scent additive from said scent additive choice, and

adding to the fragrance the selected scent additive in an amount sufficient to enhance an olfactive note in the fragrance to yield a fragrance blend having an enhanced scent,

wherein the fragrance has a first fragrance, the selected scent additive has a second fragrance, and the fragrance blend has a third fragrance.

Also provided is a fragrance blend comprising a fragrance and a scent additive. Also provided is a fragrance blend comprising a fragrance, a scent additive, and an effect additive.

Also provided is a fragrance blend prepared by any of the methods described herein.

In some embodiments, the fragrance is an existing commercial product.

In some embodiments, the fragrance is a classic product.

In some embodiments, the fragrance construct is a fragrance pyramid. In some embodiments, the fragrance construct is a top-heavy fragrance pyramid. In some embodiments, the fragrance construct is a bottom-heavy fragrance pyramid.

In some embodiments, the fragrance construct is a fragrance diamond.

In some embodiments, the fragrance is chosen from Burberry HER eau de parfum, Burberry MY BURBERRY BLACK parfum, Burberry MY BURBERRY BLUSH eau de parfum, Burberry MY BURBERRY eau de toilette, Burberry BRIT FOR HER eau de toilette; Burberry BRIT RHYTHM FLORAL eau de toilette, Burberry BODY eau de parfum, Burberry BODY TENDER eau de toilette, Burberry BODY eau de toilette, Burberry TOUCH eau de parfum, Burberry WEEKEND eau de parfum, Burberry BABY TOUCH ALCOHOL FREE, Calvin Klein WOMEN, Calvin Klein OBSESSED, Calvin Klein CK ONE, Calvin Klein CK ALL, Calvin Klein ETERNITY, Calvin Klein ETERNITY AIR for Women, Calvin Klein ETERNITY INTENSE, Calvin Klein ETERNITY NOW, Calvin Klein EUPHORIA, Calvin Klein DEEP EUPHORIA, Calvin Klein ETERNITY FOR MEN, Calvin Klein ETERNITY NOW FOR MEN, Calvin Klein ETERNITY INTENSE FOR MEN, Calvin Klein ETERNITY CALVIN KLEIN COLLECTOR'S EDITION, Calvin Klein ETERNITY AIR FOR MEN, Calvin Klein EUPHORIA FOR HIM, Gucci FLORA GORGEOUS GARDENIA eau de toilette, Gucci GUCCI BLOOM NETTARE DE FIORI eau de parfum, Gucci GUCCI BLOOM ACQUA DI FIORI eau de toilette, Gucci GUCCI BLOOM eau de parfum, Gucci GUCCI BLOOM hair mist, Gucci GUILTY ABSOLUTE POUR FEMME eau de parfum, Gucci GUCCI GUILTY OUD eau de parfum, Gucci GUCCI GUILTY EAU eau de toilette, Gucci GUCCI GUILTY INTENSE eau de parfum, Gucci GUILTY eau de toilette, Gucci FLORA BY GUCCI eau de toilette, Gucci FLORA BY GUCCI eau de parfum, Gucci GUCCI BAMBOO eau de toilette, Gucci GUCCI BAMBOO eau de parfum, Gucci RUSH eau de toilette, Gucci GUCCI INTENSE OUD eau de parfum, Boss BOSS THE SCENT INTENSE FOR HIM eau de parfum, Boss BOSS eau de toilette, Boss BOSS aftershave, Boss BOSS THE SCENT PRIVATE ACCORD FOR HER eau de parfum, Boss THE SCENT INTENSE FOR HER eau de parfum, Boss BOSS THE SCENT FOR HER eau de toilette, Boss BOSS MA VIE POUR FEMME eau de parfum, Boss BOSS MA VIE INTENSE eau de parfum, Boss BOSS MA VIE FLORALE eau de parfum, Boss BOSS MA VIE RUNWAY EDITION eau de parfum, Boss WOMAN eau de parfum, Boss JOUR LUMINEUSE eau de parfum, Boss BOSS JOUR RUNWAY EDITION eau de parfum, Boss BOSS NUIT POUR FEMME equ de parfum, Boss BOSS NUIT RUNWAY EDITION, eau de parfum, Boss FEMME BY BOSS eau de parfum, Hugo HUGO ICED eau de toilette, Boss BOSS BOTTLED UNITED eau de toilette, Hugo HUGO URBAN JOURNEY eau de toilette, Hugo HUGO MAN eau de toilette, Hugo HUGO MAN aftershave, Boss BOSS BOTTLED TONIC eau de toilette, Boss BOSS THE SCENT eau de toilette, Boss BOSS THE SCENT aftershave, Boss BOSS THE SCENT aftershave balm, Boss BOSS BOTTLED aftershave, Boss BOSS BOTTLED eau de toilette, Boss BOSS BOTTLED UNLIMITED eau de toilette, Hugo HUGO MAN EXTREME eau de parfum, Hugo HUGO ENERGISE eau de toilette, Boss BOSS BOTTLED NIGHT eau de toilette, Hugo HUGO JUST DIFFERENT eau de toilette, Boss BOSS THE SCENT PRIVATE ACCORD FOR HIM eau de toilette, Philosophy AMAZING GRACE BALLET ROSE eau de toilette, Philosophy PURE GRACE NUDE ROSE eau de toilette, Philosophy PURE GRACE eau de toilette, Philosophy LIVING GRACE eau de toilette, Philosophy FRESH CREAM eau de toilette, Philosophy UNCONDITIONAL LOVE eau de toilette, Philosophy AMAZING GRACE eau de parfum, Philosophy FALLING IN LOVE eau de toilette, Philosophy FRESH CREAM WARM eau de toilette, Philosophy SNOW ANGEL eau de toilette, Philosophy AMAZING GRACE body oil mist, Philosophy FALLING IN LOVE perfumed body spritz, Philosophy AMEN cologne, Philosophy LOVESWEPT eau de toilette, Philosophy SUNSHINE GRACE eau de toilette, Philosophy LOVESWEPT eau de parfum, and Philosophy AMAZING GRACE eau de toilette.

In some embodiments, the fragrance is chosen from the fragrances in Table 1 below.

TABLE 1

| Fragrance | Brand/Company |
|---|---|
| FARINA EAU DE COLOGNE | Johann Maria Farina |
| NUMBER SIX | Caswell-Massey |
| EAU DE LUBIN | Parfums Lubin |
| 4711 KÖLNISCH WASSER | 4711 |
| JEAN MARIE FARINA | Roger & Gallet |
| FRESHMAN | Truefitt & Hill |
| 1818 | Brooks Brothers |
| LAVENDER | Floris of London |
| POT POURRI | Officina profumo-farmaceutica di Santa Maria Novella |
| JOCKEY CLUB | Caswell-Massey |
| SANTAL IMPERIAL CREED FOR MEN | Creed |
| EAU DE COLOGNE IMÉRPIALE | Guerlain |
| FANTASIA DE FLEURS | Creed |
| HAMMAM BOUQUET | Penhaligon's |
| TABAROME (COLLECTION PRIVÉE) | Creed |
| FOUGÈRE ROYALE | Houbigant |
| AU FIL DE L'EAU | Lenthéric |
| JICKY | Guerlain |
| FLIRT | Ed. Pinaud |
| BOUQUET NOUVEAU | Roger & Gallet |
| EDWARDIAN BOUQUET | Floris of London |
| BLENHEIM BOUQUET | Penhaligon's |
| MOUCHOIR DE MONSIEUR | Guerlain |
| LA ROSE JACQUEMINOT | Coty, Paris |
| L'ORIGAN | Coty, Paris |
| AMBRE ANTIQUE | Coty, Paris |
| ENGLISH FERN | Penhaligon's |
| NARCISSE NOIR | Caron |
| L'HEURE BLEUE | Guerlain |
| QUELQUES FLEURS | Houbigant |
| UN AIR EMBAUMÉ | Rigaud |
| APRIL VIOLETS | Yardley |
| MUGUET | Coty, Paris |
| VIOLETTE PRÉCIEUSE | Caron |
| ACQUA DI PARMA COLONIA | Acqua di Parma |
| CHYPRE DE COTY | Coty, Paris |
| MITSOUKO | Guerlain |
| TABAC BLOND | Caron |
| N°5 | Chanel |
| ÉMERAUDE | Coty |
| N°22 | Chanel |
| NUIT DE NOËL | Caron |
| KRASNAYA MOSKVA | Novaya Zarya (USSR) |
| MY SIN | Lanvin |
| SHALIMAR | Guerlain |
| BOIS DES ÎLES | Chanel |
| PARIS | Coty |
| ARPÈGE | Lanvin |
| BELLODGIA | Caron |
| CUIR DE RUSSIE | Chanel |
| L'AIMANT | Coty |
| SOIR DE PARIS | Bourjois |
| LIU | Guerlain |
| ACQUA DI PARMA PROFUMO | Acqua di Parma |
| JOY | Jean Patou |
| JE REVIENS | Worth |
| TABU | Dana |
| VOL DE NUIT | Guerlain |
| ANGÉLIQUE ENCENS | Creed |
| BLUE GRASS | Elizabeth Arden |
| DUNHILL FOR MEN | Alfred Dunhill |
| POUR UN HOMME | Caron |
| NUIT DE LONGCHAMP | Parfums Lubin |
| LANCÔME | Armand Petijean |
| FRENCH CANCAN | Caron |
| KOBAKO | Bourjois |
| COLONY | Patou |
| IT'S YOU | Arden |
| SNUFF | Schiaparelli |
| CHANTILLY | Dana (Originally Houbigant) |
| BANDIT | Robert Piguet |
| FEMME | Rochas |
| WHITE SHOULDERS | Elizabeth Arden |
| COEUR-JOIE | Nina Ricci |
| MA GRIFFE | Carven |
| MISS DIOR | Christian Dior SA |
| VENT VERT | Balmain |
| FRACAS | Robert Piguet |
| L'AIR DU TEMPS | Nina Ricci |
| ROSE | Caron |
| ENGLISH LEATHER | Dana |
| ORANGE SPICE | Creed |
| EAU D'HERMÈS | Hermès |
| WIND SONG | Prince Matchabelli |
| QUADRILLE | Balenciaga |
| YOUTH DEW | Estée Lauder |
| ÉLECTRIQUE | Max Factor |
| POIVRE | Caron |
| AMBUSH | Dana |
| CHANEL POUR MONSIEUR | Chanel |
| DIORISSIMO | Christian Dior |
| LE DE | Parfums Givenchy |
| L'INTERDIT (ORIGINAL) | Parfums Givenchy |
| MONSIEUR DE GIVENCHY | Parfums Givenchy |
| CABOCHARD | Parfums Grès |
| TABAC | Maurer & Wirtz |
| UNFORGETTABLE | Avon |
| VÉTIVER | Guerlain |
| CALÈCHE | Hermès |
| DIORLING | Christian Dior |
| IDOLE DE LUBIN | Parfums Lubin |
| “Y” | Yves Saint Laurent |
| MELOGRANO | Santa Maria Novella |
| EAU SAUVAGE | Christian Dior |
| CLIMAT | Lancôme |
| IRISIA | Creed |
| Ô | Lancôme |
| CALANDRE | Paco Rabanne |
| CHAMADE | Guerlain |
| ÉQUIPAGE | Hermès |
| SÉLECTION VERTE | Creed |
| N°19 | Chanel |
| DIORELLA | Christian Dior |
| FLEURISSIMO | Creed |
| CHARLIE | Revlon |
| CIARA | Revlon |
| BABY SOFT | Love's |
| CRISTALLE | Chanel |
| EAU DE GUERLAIN | Guerlain |
| RIVE GAUCHE | Yves Saint Laurent |
| ZESTE MANDARINE PAMPLEMOUSSE | Creed |
| GUCCI POUR HOMME | Gucci |
| LILY OF THE VALLEY | Penhaligon's |
| VIOLETTA | Penhaligon's |
| Z-14 | Halston |
| LAUREN | Ralph Lauren |
| OPIUM | Yves Saint Laurent |
| ANAÏS ANAÏS | Cacharel |
| AZZARO POUR HOMME | Azzaro |
| BLUEBELL | Penhaligon's |
| CINNABAR | Estée Lauder |
| MAGIE NOIRE | Lancôme |
| WHITE LINEN | Estée Lauder |
| NAHÉMA | Guerlain |
| EAU D'ORANGE VERTE | Hermès |
| IVOIRE | Balmain |
| PATOU POUR HOMME | Jean Patou |
| KOUROS | Yves Saint Laurent |
| MUST DE CARTIER | Cartier |
| NOMBRE NOIR | Shiseido |
| ANTAEUS | Chanel |
| GIORGIO | Giorgio Beverly Hills |
| DRAKKAR NOIR | Guy Laroche |
| TRUSSARDI | Trussardi |
| PARIS | Yves Saint Laurent |
| COCO | Chanel |
| EAU POUR HOMME | Giorgio Armani |
| LUMIÈRE | Rochas |
| BEAUTIFUL | Estée Lauder |
| OBSESSION | Calvin Klein |
| POISON | Christian Dior |
| YSATIS | Parfums Givenchy |
| GREEN IRISH TWEED | Creed |
| LE JARDIN D'AMOUR | Max Factor |
| ZINO DAVIDOFF | Davidoff |
| XERYUS | Parfums Givenchy |
| PRESCRIPTIVES CALYX | Prescriptives |
| LOULOU | Cacharel |
| MA LIBERTÉ | Jean Patou |
| COOL WATER | Davidoff |
| KNOWING | Estée Lauder |
| ETERNITY | Calvin Klein |
| EXCLAMATION FOR WOMEN | Coty |
| FAHRENHEIT | Christian Dior |
| ASPEN | Coty |
| PRASTARA | Douglas Hopkins and Co. |
| RED DOOR | Elizabeth Arden |
| SAMSARA | Guerlain |
| NAVY | Dana (Orig. by CoverGirl) |
| GLOBE | Rochas |
| PREFERRED STOCK | Coty |
| TRÉSOR | Lancôme |
| ÉGOÏSTE | Chanel |
| SAFARI | Ralph Lauren |
| BALENCIAGA POUR HOMME | Balenciaga |
| VIP SPÉCIAL RÉSERVE POUR HOMME | Giorgio Beverly Hills |
| 1881 | Cerruti |
| TERRE DU SUP | Michel Klein |
| KENZO POUR HOMME | Kenzo |
| DUNE | Christian Dior |
| AMARIGE | Parfums Givenchy |
| ESCAPE | Calvin Klein |
| GUESS BY GUESS | Guess |
| PETIT GUERLAIN FOR CHILDREN | Guerlain |
| L'EAU DE BULGARI | Bulgari |
| ANGEL | Thierry Mugler |
| BOIS DE VIOLETTE | Serge Lutens |
| HÉRITAGE | Guerlain |
| L'EAU D'ISSEY | Issey Miyake |
| FÉMINITÉ DU BOIS | Shiseido |
| EROLFA | Creed |
| MINOTAURE | Parfums Paloma Picasso |
| EAU PARFUMÉE AU THÉ VERT | Bulgari |
| EAU DE ROCHAS HOMME | Rochas |
| AMBRE SULTAN | Serge Lutens |
| JEAN-PAUL GAULTIER CLASSIQUE | Jean Paul Gaultier |
| ÉQUATEUR | Bourjois |
| AMBRE | Molinard |
| XS POUR ELLE | Paco Rabanne |
| YVRES SE | YSL |
| SUBLIME FOR WOMEN | Jean Patou |
| SWEET | Courreges |
| FLEUR D'INTERDIT | Parfums Givenchy |
| KASHÂYA | Kenzo |
| CK ONE | Calvin Klein |
| TOCADE | Rochas |
| SEXUAL | Michel Germain |
| ÉDEN | Cacharel |
| BY | Dolce & Gabbana |
| VAN CLEEF | Van Cleef & Arpels |
| L'EAU D'ISSEY POUR HOMME | Issey Miyake |
| VANILLA MUSK | Coty, Inc. |
| LAILA EAU DE PARFUM | Laila The Essence of Norway |
| 24, FAUBOURG | Hermès |
| POÊME | Lancôme |
| HUGO | Hugo Boss |
| MILLÉSIME IMPÉRIAL | Creed |
| TOUCH ME | Nabeel |
| SO PRETTY | Cartier |
| KOUROS FRAICHEUR | Yves Saint Laurent |
| ACQUA DI GIÒ POUR HOMME | Giorgio Armani |
| LE MÂLE | Jean Paul Gaultier |
| ANGEL MEN/A*MEN | Thierry Mugler |
| DOLCE VITA | Christian Dior |
| SPRING FLOWER | Creed |
| ALLURE | Chanel |
| TOMMY GIRL | Tommy Hilfiger |
| ORGANZA | Parfums Givenchy |
| EAU SVELTE | Dior |
| ENCENS ET LAVANDE | Serge Lutens |
| L'EAU PAR KENZO | Kenzo |
| ENVY | Gucci |
| LOLITA LEMPICKA | Lolita Lempicka |
| DALIMIX GOLD | Salvador Dali |
| OURAGAN | Bourjois |
| LOVE STORY | Louis Féraud |
| IN LOVE AGAIN | Yves Saint Laurent |
| UOMO | Moschino |
| V/S | Versace |
| HYPNOTIC POISON | Christian Dior |
| ALCHIMIE | Rochas |
| OPIUM FRAÎCHEUR D'ORIENT | Yves Saint Laurent |
| BULGARI BLACK | Bulgari |
| NOA | Cacharel |
| GUERLINADE | Guerlain |
| II | Parfums Givenchy |
| ACQUA ALLEGORIA | Guerlain |
| JUNGLE HOMME | Kenzo |
| UNE FLEUR | Chanel |
| ODEUR 53 | Comme des Garc̨ons |
| UN AMOUR | Jean Patou |
| ENVY FOR MEN | Gucci |
| ROMANCE | Ralph Lauren |
| LE BAISER DE LALIQUE | Lalique |
| HIRIS | Hermès |
| DZING! | L'Artisan |
| J'ADORE | Christian Dior |
| PASSAGE D'ENFER | Comme des Garçons |
| GREEN TEA | Elizabeth Arden |
| BABY DOLL | YSL |
| ALLURE HOMME | Chanel |
| FRAGILE | Jean Paul Gaultier |
| GUET APENS | Guerlain |
| LACOSTE FOR WOMEN | Lacoste |
| ANNA SUI CLASSIC | Anna Sui |
| MASCULIN EXTRÊME | Bourjois |
| EN PASSANT | Frédéric Malle |
| L'ANARCHISTE | Caron |
| TRUTH | Calvin Klein |
| DILMUN LORENZO VILLORESI PERFUME | Lorenzo Villoresi |
| TEA FOR TWO | L'Artisan Parfumeur |
| WINTER DELICE | Guerlain |
| BELLE DE MINUIT | Nina Ricci |
| BODY POWER | Estée Lauder |
| FLOWER BY KENZO | Kenzo |
| CHERRY BLOSSOM | Guerlain |
| RÊVERIE | Gloria Vanderbilt |
| ROUGE HERMÈS | Hermès |
| INITIAL | Boucheron |
| PHILTRE D'AMOUR | Guerlain |
| SUI DREAMS | Anna Sui |
| MAHORA | Guerlain |
| 1872 | Clive Christian |
| CHERGUI | Serge Lutens |
| COCO MADEMOISELLE | Chanel |
| LIGHT BLUE | Dolce & Gabbana |
| MUGLER COLOGNE | Thierry Mugler |
| NO. 1 | Clive Christian |
| NU | Yves Saint Laurent |
| X | Clive Christian |
| EAU DE CARTIER | Cartier |
| HIGHER | Christian Dior |
| PANAME | Jean Patou |
| KENZOKI ÉNERGISANT | Kenzo |
| LACOSTE POUR HOMME | Lacoste |
| ADDICT | Christian Dior |
| BLACK CASHMERE | Donna Karan |
| 2 | Comme des Garçons |
| CHANCE | Chanel |
| FIERCE | Abercrombie & Fitch |
| M7 | Yves Saint Laurent |
| ABSOLU | Rochas |
| GLORIA | Cacharel |
| AQUAWOMAN | Rochas |
| SUI LOVE | Anna Sui |
| ESSENZA DI ZEGNA | Ermenegildo Zegna |
| VERSACE MAN | Versace |
| AMOR AMOR | Cacharel |
| BEYOND PARADISE | Estée Lauder |
| LIFE | Armanis |
| EAU DE CRISTOBAL | Balenciaga |
| FOLIFLORA | Guerlain |
| BRIT | Burberry |
| D | Diane von Fürstenberg |
| 100% LOVE | S-Perfume |
| OMNIA | Bulgari |
| NARCISO RODRIGUEZ FOR HER | Narciso Rodriguez |
| EAU PARFUMÉE AU THÉ BLANC | Bulgari |
| ECHO | Davidoff |
| GRAMERCY PARK | Bond No. 9 |
| DOLLY GIRL | Anna Sui |
| BOSS BLUE EDITION | Hugo Boss |
| EAU DES MERVEILLES | Hermès |
| BE DELICIOUS | Donna Karan |
| L'INSTANT DE GUERLAIN POUR HOMME | Guerlain |
| APPARITION | Ungaro |
| FLOWERBOMB | Viktor & Rolf |
| PURE POISON | Christian Dior |
| AQUA ALLEGORIA ANISIA BELLA | Guerlain |
| ARMANI CODE | Giorgio Armani |
| ALLURE HOMME SPORT | Chanel |
| BEYOND PARADISE POUR HOMME | Estée Lauder |
| B MEN | Thierry Mugler |
| BOIS D'ENCENS | Armani Privé |
| DOLLY GIRL OOH LA LOVE | Anna Sui |
| GAULTIER | Jean Paul Gaultier |
| ALIEN | Thierry Mugler |
| CHINATOWN | Bond No. 9 |
| LOVE IN WHITE CREED | Creed |
| EUPHORIA | Calvin Klein |
| AQVA POUR HOMME | Bulgari |
| UN JARDIN SUR LE NIL | Hermès |
| SILVER BLACK | Azzaro |
| FLOWERBOMB | Viktor & Rolf |
| HYPNÔSE | Lancôme |
| SECRET WISH | Anna Sui |
| BLACK ORCHID | Tom Ford |
| BVLGARI POUR HOMME SOIR | Bulgari |
| ENCRE NOIRE | Lalique |
| TERRE D'HERMÈS | Hermès |
| LILY & SPICE | Penhaligon's |
| CRYSTAL NOIR | Versace |
| INSOLENCE | Guerlain |
| L'HOMME | Yves Saint Laurent |
| PRINCESS | Vera Wang |
| L | Lolita Lempicka |
| AMBER POUR HOMME | Prada |
| ANGE OU DÉMON | Parfums Givenchy |
| DOLLY GIRL ON THE BEACH | Anna Sui |
| SECRET WISH: MAGIC ROMANCE | Anna Sui |
| FIORUCCI GLITTERY | Fiorucci |
| LIGHT BLUE POUR HOMME | Dolce & Gabbana |
| FLEUR DU MÂLE | Jean Paul Gaultier |
| PRADA INFUSION D'IRIS | Prada |
| GUCCI BY GUCCI | Gucci |
| BLACK | Roberto Cavalli |
| MIDNIGHT POISON | Dior |
| XX | Hugo Boss |
| ICE MEN | Thierry Mugler |
| ABSOLUTELY GIVENCHY | Parfums Givenchy |
| 28 LA PAUSA | Chanel |
| DAISY | Marc Jacobs |
| FUEL FOR LIFE | Diesel |
| DOLLY GIRL BONJOUR L'AMOUR | Anna Sui |
| FLIGHT OF FANCY | Anna Sui |
| AGELESS | Harvey Prince |
| FLEUR DÉFENDUE | Lolita Lempicka |
| FÉERIE | Van Cleef & Arpels |
| 8 88 | Comme des Garçons |
| GUCCI BY GUCCI - POUR HOMME | Gucci |
| DIAMONDS | Emporio Armani |
| ADVENTURE | Davidoff |
| ONDE | Armani |
| THE ONE | Dolce & Gabbana |
| ELIXIR | Penhaligon's |
| LONDON | Dunhill |
| FUEL FOR LIFE | Diesel |
| BLUE MEDITERRANEO - MIRTO DI PANAREA | Acqua di parma |
| GUERLAIN HOMME | Guerlain |
| L'HOMME | Guerlain |
| 1881 EN FLEURS | Cerruti |
| DOLLY GIRL LIL' STARLET | Anna Sui |
| NIGHT OF FANCY | Anna Sui |
| TEMPTRESS | Harvey Prince |
| VERSENSE | Versace |
| BLUE BLOODED | Amordad |
| ONLY THE BRAVE | Diesel |
| UOMO | Ferrari |
| ACQUA FIORENTINA | Creed |
| LA NUIT DE L'HOMME | Yves Saint Laurent |
| L'EAU DE GENTIANE BLANCHE | Hermès |
| LIVE YOUR DREAM | Anna Sui |
| ROCK ME! | Anna Sui |
| YOGINI | Harvey Prince |
| LOVE, CHLOÉ | Chloé |
| TONKA IMPERIALE | Guerlain |
| VOYAGE D'HERMÈS | Hermès |
| AVENTUS | Creed |
| BLEU DE CHANEL | Chanel |
| WOMANITY | Thierry Mugler |
| FORBIDDEN AFFAIR | Anna Sui |
| ROCK ME! SUMMER OF LOVE | Anna Sui |
| 1697 FRAPIN | Frapin |
| CK ONE SUMMER 2011 | Calvin Klein |
| NUIT | Christian Lacroix |
| THESEUS LORENZO VILLORESI FIRENZE | Lorenzo Villoresi |
| LE PARFUM | Elie Saab |
| KOKORICO | Jean Paul Gaultier |
| THIERRY MUGLER ALIEN SUNESSENCE | Thierry Mugler |
| EDITION LIMITEE 2011 OR D'AMBRE | |
| ESPECIALLY ESCADA | Escada |
| FAN DI FENDI | Fendi |
| MICHAEL KORS SUEDE | Michael Kors |
| ORIFLAME LOVELY GARDEN | Oriflame |
| MI CORAZON | Oscar de la Renta |
| CORALINA | Oscar de la Renta |
| SARGASSO | Oscar de la Renta |
| ORIENTAL LACE | Oscar de la Renta |
| SANTO DOMINGO | Oscar de la Renta |
| GRENADA | Oscar de la Renta |
| ETERNITY AQUA FOR WOMEN CALVIN | Calvin Klein |
| KLEIN FOR WOMEN | |
| FAME THE FIRST EVER BLACK EAU DE PERFUME | Lady Gaga |
| FLORENTINE IRIS ESSENZE | Ermenegildo Zegna |
| SECRET WISH FAIRY DANCE | Anna Sui |
| CHARRIOL ROYAL WHITE | Charriol |
| L'EAU ROSE | Balenciaga |
| ESTÉE LAUDER AMBER MYSITIQUE | Estée Lauder |
| GUERLAIN AQUA ALLEGORIA NEROLIA BIANCA | Guerlain |
| JASMIN PERLE DE THÉ | Fragonard |
| UNTOLD | Elizabeth Arden |
| FLASH | Jimmy Choo |
| GUESS GIRL | Guess |
| LA VIE DE BOHÈME | Anna Sui |
| TIN HOUSE FAIRY DANCE | Anna Sui |
| TIN HOUSE FLIGHT OF FANCY | Anna Sui |
| TIN HOUSE FORBIDDEN AFFAIR | Anna Sui |
| TIN HOUSE SECRET WISH | Anna Sui |
| HELLO | Harvey Prince |
| PETALLY NOIR | Harvey Prince |
| VINCE CAMUTO FOR MEN | Vince Camuto |
| REVEAL | Calvin Klein |
| CUIR CANNAGE | Dior |
| L'OCCITANE EN PROVENCE AGUAPE | L'Occitane en Provence |
| LA NUIT DE BOHÈME | Anna Sui |
| LA NUIT DE BOHÈME EAU DE PARFUM | Anna Sui |
| SUI DREAMS IN PINK | Anna Sui |
| MAGNIFICENT BLOSSOM | Yves Saint Laurent |
| ROMANTICA | Anna Sui |
| SUI DREAMS IN GREEN | Anna Sui |
| DUBAI MEYDAN | Nabeel |
| FRAGONARD IRIS | Fragonard |
| SUI DREAMS IN YELLOW | Anna Sui |
| PURE GOLD | Pure Gold perfumes |
| LUCKY WISH | Anna Sui |
| ROMANTICA EXOTICA | Anna Sui |
| MUGUET 2016 GUERLAIN | Guerlain |
| FALCONER | Ottaniqo |
| L'AMOUR ROSE | Anna Sui |

In some embodiments, the plurality of scent additives comprises musk oil, civet, castoreum, ambergris, plant perfumes, sandalwood oil, neroli oil, bergamot oil, lemon oil, lavender oil, sage oil, rosemary oil, peppermint oil, eucalyptus oil, menthol, camphor, verbena oil, citronella oil, coconut oil, salvia oil, clove oil, chamomile oil, costus oil, labdanum oil, broom extract, carrot seed extract, jasmine extract, mimosa extract, narcissus extract, olibanum extract, rose extract, acetophenonene and derivatives thereof, dimethylaniline derivatives, naphthaline derivatives, allyl caprate, alpha-amylcinnamic aldehyde, anethole, anisaldehyde, benzyl acetate, benzyl alcohol, benzyl propionate, borneol, cinnamyl acetate, cinnamyl alcohol, citral, citronellal, cumin aldehyde, cyclamen aldehyde, decanol, ethyl butyrate, ethyl caprate, ethyl cinnamate, ethyl vanillin, eugenol, geraniol, exenol, alpha-hexylcinnamic aldehyde, hydroxycitrolnellal, indole, iso-amyl acetate, iso amyl isovalerate, iso-eugenol, linalol, linalyl acetate, p-methylacetophenone, methyl anthranilate, methyl dihydrojasmonate, methyl eugenol, methyl-beta-naphthol ketone, methyl phenyl carbinyl acetate, musk ketol, musk xylol, 2,5,6-nonadienal, gamma-nonalactone, phenyl acetaldehyde dimethyl acetal, beta-phenylethyl alcohol, 3,3,5-trimethylcyclohexanol, gamma-undecalactone, undecenal, vanillin, metofluthrin, limonene, terpinene gamma, calone, helional, and mixtures thereof.

In some embodiments, the plurality of scent additives comprises musk oil, civet, castoreum, ambergris, plant perfumes, sandalwood oil, neroli oil, bergamot oil, lemon oil, lavender oil, sage oil, rosemary oil, peppermint oil, eucalyptus oil, menthol, camphor, verbena oil, citronella oil, coconut oil, salvia oil, clove oil, chamomile oil, costus oil, labdanum oil, broom extract, carrot seed extract, jasmine extract, mimosa extract, narcissus extract, olibanum extract, rose extract, acetophenonene and derivatives thereof, dimethylaniline derivatives, naphthaline derivatives, allyl caprate, alpha-amylcinnamic aldehyde, anethole, anisaldehyde, benzyl acetate, benzyl alcohol, benzyl propionate, borneol, cinnamyl acetate, cinnamyl alcohol, citral, citronellal, cumin aldehyde, cyclamen aldehyde, decanol, ethyl butyrate, ethyl caprate, ethyl cinnamate, ethyl vanillin, eugenol, geraniol, exenol, alpha-hexylcinnamic aldehyde, hydroxycitrolnellal, indole, iso-amyl acetate, iso amyl isovalerate, iso-eugenol, linalol, linalyl acetate, p-methylacetophenone, methyl anthranilate, methyl dihydrojasmonate, methyl eugenol, methyl-beta-naphthol ketone, methyl phenyl carbinyl acetate, musk ketol, musk xylol, 2,5,6-nonadienal, gamma-nonalactone, phenyl acetaldehyde dimethyl acetal, beta-phenylethyl alcohol, 3,3,5-trimethylcyclohexanol, gamma-undecalactone, undecenal, vanillin, metofluthrin, and mixtures thereof.

In some embodiments, the plurality of scent additives comprises at least two of bergamot oil, jasmine extract, lavender oil, limonene, terpinene gamma, calone, helional, and mixtures thereof.

In some embodiments, the plurality of scent additives comprises limonene, terpinene gamma, calone, helional, and mixtures thereof.

In some embodiments, the plurality of scent additives comprises bergamot oil, jasmine extract, lavender oil, and mixtures thereof.

In some embodiments, the plurality of scent additives comprises bergamot oil, jasmine extract, and lavender oil.

In some embodiments, the plurality of scent additive comprises one or more perfuming ingredients having a contrasting notes to the one or more olfactive notes of the fragrance.

In some embodiments, the plurality of scent additive comprises one or more perfuming ingredients having one or more olfactive notes of the fragrance.

In some embodiments, selected scent additive is a perfuming ingredient having a contrasting note to one or more olfactive notes of the fragrance.

In some embodiments, the selected scent additive is a perfuming ingredient having one or more olfactive notes of the fragrance.

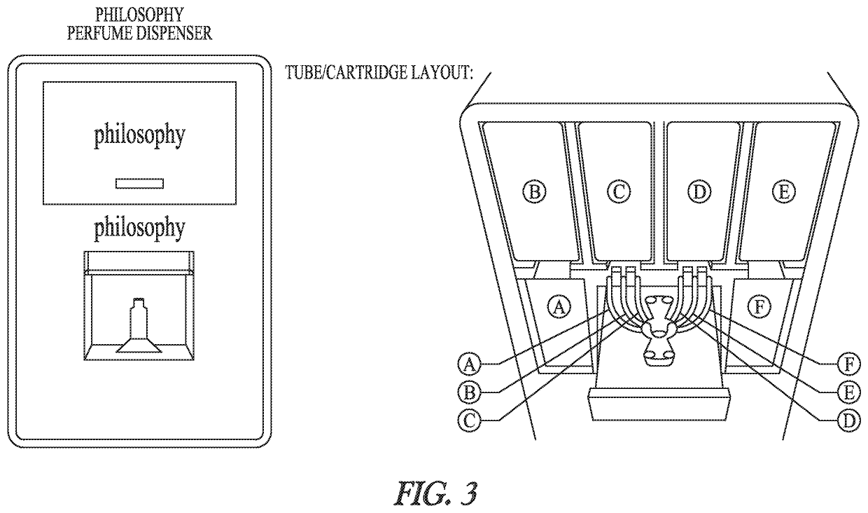

In some embodiments, adding the selected scent additive to the fragrance is accomplished is performed manually. In some embodiments, adding the selected scent additive to the fragrance is accomplished is performed using an automated perfume dispenser, such as that shown in FIG. 3. See, e.g., PCT Publication No. WO 2018/162883, PCT Publication No. WO 2015/082917, and U.S. Patent Application No. 62/632,191, filed Feb. 19, 2018.

In some embodiments, the method further comprises determining how much of the scent additive to add to the fragrance.

In some embodiments, the scent additive is present in an amount sufficient to enhance a scent in the fragrance. In some embodiments, the scent additive is present in the fragrance blend in an amount of at least 45%. In some embodiments, the scent additive is present in the fragrance blend in an amount of between about 0.1% and about 65%, such as between about 0.1% and about 50%, for example, between about 0.1% and about 40%, such as between about 0.1% and 30%, for example, between about 0.1% and about 20%, such as between about 0.1% and 15%, such as between about 0.11% and 12%. Those with skill in the art will be able to employ the desired amount to provide desired fragrance effect and intensity.

In some embodiments, the fragrance blend is formulated as a perfume, perfume water, Eau de toilette, Eau de cologne, Eau de parfum, fragrance mist, or air freshener.

In some embodiments, the fragrance blend is formulated as a body lotion, body crème, deodorant spray, deodorant stick, shower gel, body scrub, shampoo, bubble bath, or soap.

In some embodiments, the method further comprises:

presenting the individual with an effect additive choice comprised of a plurality of effect additives;

receiving from the individual a selection of an effect additive from said plurality of effect additives, and

adding to the fragrance the selected effect additive.

In some embodiments, the effect additive choice comprises particles that impart a tactile sensory experience to an end user. In some embodiments, the particles impart a soft sensory experience. In some embodiments, the particles impart a cool or warm sensory experience. In some embodiments, the particles impart a conditioner to the skin of a user.

In some embodiments, the effect additive choice comprises additives that provide a visual effect. In some embodiments, the visual effect comprises a sparkle from light reflecting, glittering particles. In some embodiments, the light reflecting glittering particles are selected from one or more of titanium oxide, mica, other iron oxides such as yellow, red and brown, black and tin oxide. In some embodiments, the visual effect comprises particles that include one or more of ferric ammonium ferrocyanide (blue), manganese violet, ultramarine blue, chrome oxide (green), 30 talc, lecithin modified talc, zeolite, kaolin, lecithin modified kaolin, titanium dioxide (white) and mixtures thereof. In some embodiments, the visual effect comprises one or more pearlescents such as mica, bismuth oxychloride and treated micas, such as titanated micas and lecithin modified micas that include mica, coated with varying thickness of titanium dioxide and particles that include thin film optics imparting brilliant luster and a broad range of interference colors and multicolor effect. In some embodiments, the visual effect comprises one or more particles that include tiny light emitting diodes, LED's, or luminescent materials that impart to the fragrance a capacity for emitting light, either continuously or intermittently.

In some embodiments, the effect additive choice comprises one or more modulators optionally with one or more co-modulators. In some embodiments, the one or more modulators is chosen from 3-((2-ethylhexyl)oxy)-propane-1,2-diol; Dibenzylidene Sorbitol; Dislearyl Ether; Ethylene glycol monomethyl ether; Tri-o-(cis-3-hexenyl)-glactopyranose; (E)-1-(2,2,6-trimethylcyclohexyl)oct-1-en-3-one; 1-amino-3-((2-ethylhexyl)oxy)-propan-2-ol; 1-(1-Methyl-2-propoxyelhoxy)-2-propanol; 1-(1-menthoxy)propane-2-ol; 1,1,2,4,4-pentamethyl-6-acetyl-7-formyl-1,2,3,4-tetrahydronaphthalene; 1,1,4,4-tetramethyl-6-acetyl-7-formyl-1,2,3,4-tetrahydronaphthalene; 2-(2-butoxyethoxy)ethanol; 2-(2-Butoxyethoxy)ethyl (6-propylpiperonyl) ether; 2-(Hexadecyloxy)ethanol; 2-(1-menthoxy)ethane-1-ol; 2,4,6-Tris[bis(methoxymethyl) amino]-1,3,5-triazine formaldehyde)methylated; 2-Diphenylmethoxy-N,N-dimethylethylamine hydrochloride; 2-methyl-3-(1-menthoxy)propane-1,2-diol; 3-((2-propylheptyl)oxy)-propane-1,2-diol; 3-(1-menthoxy)propane-1,2-diol; 3-(1-menthoxy)propane-1-ol; 3-Methoxybutanol; 4-(1-menthoxy) butane-1-ol; Alcohols, C12-14, ethoxylated; Alkoxylaled Alcohols; Allantoin Ascorbate; Batyl Alcohol; Beheneth-5; Benzylhemiformal; Bis-methoxy PEG-13.PEG-438/PPG-110 SMDI Copolymer; Bis-O-(3,6-dioxadecanyl)-glucopyranose; Bis-O-(3,6-dioxadodecanyl)-glucopyranose; Bis-O-(3,6-dioxaheptanyl)-xylopyranose; Bis-O-(3,6-dioxaoctanyl)-galactopyranose; Butoxyethanol; C10-16 Pareth-1; C12-14 Sec-Pareth-3; C14-15 Pareth-7; Caprylyl Glyceryl Ether; Cetearyl Ethylhexnoate; Ceteth-10; Ceteth-12; Ceteth-2; Ceteth-3; Ceteth-4; Ceteth-5; Cetoleth-10; Cetoleth-10; Chimyl Alcohol; Choleth-10; Coceth-10 or Polyoxyethylene (10) dodecyl ether; Decaethylene glycol mono-dodecyl ether lauryl ether; Deceth-4; Deceth-4 Phosphate; Deceth-6; Dermofeel G 10 L; Dicetyl Ether; Dielhylene Glycol; Diethoxynonadiene; Diethylene glycol ethyl ether; Diglycerin; Dihydro-7a-ethyloxazolo [3,4-c]oxazole dioxabicyclo[3.3.0]octane; Diisoamyl Adipate; Diisobutyl Adipate; Dimethoxydiglycol Ethers; Dimethyl Oxazolidine; Di-O-(cis-3-hexenyl)-glactopyranose; Di-O-(linalyl)-glucopyranose; Dioxolane; Dipentaerythrityl Hexacaprylate/Hexacaprate; Dipropylene Glycol Dimethyl Ether; Dipropylene glycol monomethyl ether; Disodium Lauroamphodipropionate; Distearyl Ether; DL-Panthenol; Ethoxyethanol Alcohols; Diethyl Ether; Ethyl Hydroxymethyl Oleyl Oxazoline; Ethylhexylglycerin; Glycerol ethoxylate; Glycerol Formal; Glycerol propoxylate; Hexaethylene glycol monododecyl ether; Hexaethylene glycol monohexadecyl ether; Hexaethylene monododecyl ether; Hyaluronic acid disaccharide sodium salt; Hydroquinone beta-D-glycoside; Hydroxymethyl Dioxoazabicyclooctane; Isobutoxypropanol Alcohols; Isoceteth-10; Isoceteth-12; Isoceteth-15; Isoceteth-20; Isoceteth-25; Isoceteth-30; Isoceteth-5; Isoceteth-7; Isosteareth-5; Laureth-1; Laureth-10; Laureth-2; Laureth-3; Laureth-4; Laureth-5; Laureth-7; Laureth-8; Linear Alcohol (C11) Elhoxylate, POE-5; Linear alcohol (C12-13) Pareth-3ethoxylated; Linear alcohol (C12-15) Pareth-3 ethoxylate; Linear alcohol (C9-11) ethoxylated POE-8 Pareth-3; Menthone Glycerin Acetal; Menthoxypropanediol; Methoxydiglycol Ethers; Methoxyethanol; Methoxyisopropanol Alcohols; Methyl Hexyl Ether Ethers; Methyl Hydroxymethyl Oleyl Oxazoline; Methyl Morpholine oxide Amine Oxides; Methylal Ethers; Mono-O-(cis-3-hexenyl)-glactopyranose; Mono-O-(linalyl)-glucopyranose; Morpholine; Myreth-3; Myreth-4; Myreth-5; Neopentyl Glycol Diisononanoate; N-hexadecyl n-nonanoate (i.e., cetyl nonanoate)/Noctadecyl n-nonanoate (i.e., stearyl nonanoate); Nonaethylene glycol monododecyl ether monododecyl ether; Nonoxynol-10; Nonoxynol-2; Nonoxynol-3; Nonoxynol-4; Nonoxynol-5; Nonoxynol-6; Nonoxynol-7; Nonoxynol-8; Nonoxynol-9; Nonoxynol-9 Iodine; Octaethylene Glycol; Octoxynol-10; Octoxynol-3; Octyl ether Dioctyl ether; Octylphenoxy poly(ethyleneoxy)ethanol, branched; Oleth-10; Oleth-16 Alkoxylated Alcohols; Oleth-2; Oleth-3; Oleth-4; Oleth-5; Oleth-8; Panthenol Ethyl Ether; PEG-10; PEG-12; PEG-15 Oleamine; PEG-3 Methyl Ether; PEG-5 Hydrogenated Tallow Amine; PEG-6; PEG-9; Pentakis-O-(3,6-dioxadecanyl)-glucopyranose; Pentakis-O-(3,6-dioxadodecanyl)-glucopyranose; Pentakis-O-(3,6-dioxaoctanyl)-galactopyranose; Penta-O-(cis-3-hexenyl)-glactopyranose; Penta-O-(linalyl)-glucopyranose; Pluronic F-127; Poloxamine 1307; Poly(ethylene glycol) methyl ether; Poly(ethylene glycol-ran-propylene glycol) monobutyl ether; Poly(oxy-1,2-ethanediyl), a-decyl-w-hydroxy-Ethoxylate; Poly(propylene glycol) monobutyl ether; Polyethylene glycol 1100 mono(hexadecyl/octadecyl); Polyethylene glycol hexadecyl ether; Polyglycerin #310; Polyglycerin-4 Ethers; Polyglyceryl-10 Dioleate; Polyglyceryl-10 Myristale; Polyglyceryl-10 Oleate; Polyglyceryl-10 Stearate; Polyglyceryl-10 Tetraoleale; Polyglyceryl-10 Trioleate; Polyglyceryl-2 Oleyl Ether; Polyglyceryl-6 Isostearate; Polyglyceryl-6 Laurate; Polyimide-1; Polymethoxy Bicyclic Oxazolidine; Polyoxy(ethylene glycol); Polyoxyethylene (20) oleyl ether; Polyquaternium-10; Polyvinyl Stearyl Ether; PPG-10 Cetyl Ether; PPG-11 Stearyl Ether; PPG-11 Stearyl Ether Kj; PPG-2 Methyl Ether; PPG-23-Steareth-34; PPG-3 Methyl Ether Alkoxylaled Alcohols; PPG-4-Ceteth-10; PPG-5-Ceteth-20; Pramoxine HCl; propyl {4-[2-(diethylamino)-2-oxoethoxy]-3-methoxyphenyl) acetate; Propylene Glycol; Propylene Glycol Caprylate; Propylene Glycol n-Butyl Ether; Propylene Glycol Propyl Ether; Sodium Hyaluronate; Sodium Octoxynol-2 Ethane Sulfonate; Stearamidopropyl Morpholine; Stearamidopropyl Morpholine Lactate; Steareth-10; Steareth-10 Allyl Ether; Steareth-10 Allyl Ether/Acrylates Copolymer; Steareth-2; Steareth-3; Steareth-4; Steareth-5; Steareth-6; Steareth-7; Stearoxypropyl Dimethylamine; Sucrose dilaurate; Sucrose Distearate; Sucrose laurate; Sucrose myristate; Sucrose palmitate; Sucrose stearate; Sucrose Tristearate; Talloweth-7; TEA-Lauryl Ether; Tetra(ethylene glycol); Tetrakis-O-(3,6-dioxadodecanyl)-glucopyranose; Tetrakis-O-(3,6-dioxaheptanyl)-xylopyranose; Tetrakis-O-(3,6-dioxaoctanyl)-galactopyranose; Tetra-O-(cis-3-hexenyl)-glactopyranose; Tetra-O-(linalyl)-glucopyranose; Tri(propylene glycol) n-butyl ether; Trideceth-3; tridecyl ether; Triethylene glycol; Triglycerol diisostearate; Trimethyl Pentanol Hydroxyethyl Ether; Tri-O-(linalyl)-glucopyranose; Tripropylene Glycol; Tris-O-(3,6-dioxadecanyl)-glucopyranose; Tris-O-(3,6-dioxadodecanyl)-glucopyranose; Tris-O-(3,6-dioxaheptanyl)-xylopyranose; and Tris-O-(3,6-dioxaoctanyl)-galactopyranose.

In some embodiments, the one or more modulators is an alkoxylated glucoside which can be selected, for example, from methyl glucoside polyol, ethyl glucoside polyol, and propyl glucoside polyol. In one example, the alkoxylated glucoside is PPG-20 Methyl Glucose Ether (GLUCAM P-20 available from Lubrizol, USA).

In some embodiments, the one or more modulators and optionally, the one or more co-modulators, are independently chosen from the modulators and co-modulators described in U.S. Pat. No. 9,814,661, US Patent Publication 20160362629, US Patent Publication 20160376525, US Patent Publication 20160362631, and US Patent Publication 20160362630.

In some embodiments, the effect additive choice comprises one or more entrapment materials. Suitable entrapment materials for use herein are selected from polymers; capsules, microcapsules and nanocapsules; liposomes, absorbents; cyclic oligosaccharides and mixtures thereof. Cyclic oligosaccharides can include PCT Publication Nos. WO2000/67721 (Procter & Gamble); and WO2000/67720 (Procter & Gamble); and U.S. Pat. No. 6,893,647 (Procter & Gamble)). The cyclic oligosaccharide may comprise any suitable saccharide or mixtures of saccharides. Examples of suitable saccharides include, but are not limited to, glucose, fructose, mannose, galactose, maltose and mixtures thereof. A cyclic oligosaccharide comprises glucose. The cyclic oligosaccharides for use herein can include α-cyclodextrins or β-cyclodextrins, or mixtures thereof. The cyclic oligosaccharide, or mixture of cyclic oligosaccharides, for use herein may be substituted by any suitable substituent or mixture of substituents. Examples of cyclic oligosaccharides for use herein are α-cyclodextrin, β-cyclodextrin, methyl-α-cyclodextrin, methyl-β-cyclodextrin, hydroxypropyl-α-cyclodextrin and hydroxypropyl-β-cyclodextrin. A person skilled in the art will recognize that the levels of cyclic oligosaccharides will also be dependent on the components of the composition and their levels, for example the solvents used or the exact fragrance oils, or combination of fragrance oils, present in the composition.

In some embodiments, the effect additive choice comprises an anti-habituation material. In some embodiments, the anti-habituation material is chosen a) from about 0.0000001% to about 10% of a perfume raw material comprising a thiol moiety; b) from about 0.0000001% to about 10% of a perfume raw material comprising a sulfide moiety; c) from about 0.000001% to about 10% of a perfume raw material comprising a thiazole moiety; d) from about 0.0000001% to about 10% of a perfume raw material comprising an oxathiane moiety; e) from about 0.000001% to about 10% of a perfume raw material comprising oxygen, sulfur, and nitrogen; f) from about 0.00000005% to about 5% of a perfume raw material comprising a pyrazine moiety; g) from about 0.00001% to about 20% of a perfume raw material comprising a nitrile moiety; h) from about 0.000001% to about 10% of a perfume raw material comprising an indole moiety; i) from about 0.00001% to about 10% of a perfume raw material comprising an oxime moiety; j) from about 0.00001% to about 20% of a perfume raw material comprising an amine moiety; k) from about 0.00000005% to about 5% of a perfume raw material comprising an isothiocyanate; 1) from about 0.00000005% to about 5% of a perfume raw material comprising a diamine moiety; and m) mixtures thereof, with the proviso that the sum of the percentage of the perfume raw materials cannot exceed 100%. In some embodiments, the effect additive choice is chosen from the anti-habituation materials described in US Patent Publication No. 2015322376.

In some embodiments, the scent additive and effect additive is present in the fragrance blend in an amount of at least 45%, such as at 45-90%, e.g., 50-75% or 50-60%. Those with skill in the art will be able to employ the desired amount to provide desired fragrance effect and intensity.

Also provided is a fragrance blend prepared by any of the methods described herein.

Also provided is a fragrance blend comprising a fragrance and a scent additive.

In some embodiments, the fragrance, scent additive, and optional effect additive are dispensed into a container. In some embodiments, the fragrance, scent additive, and optional effect additive are dispensed into a bottle. In some embodiments, the fragrance, scent additive, and optional effect additive are dispensed into a bottle with a screw on spray pump. In some embodiments, the fragrance, scent additive, and optional effect additive are dispensed into a bottle with a spray pump that is crimped onto the bottle.

In some embodiments, the container, such as a bottle with a screw on spray pump or a crimped spray pump, can be re-filled with the same or another fragrance blend. See, e.g., US2018141066, FR3049267, FR3037577, U.S. Ser. No. 10/399,103, and FR3008901.

In some embodiments, the method further comprises provided a sample of the fragrance blend to the individual.

In some embodiments, the individual is satisfied with the sample, and the method further comprises providing the fragrance blend to the individual.

In some embodiments, the individual is not satisfied with the sample, and the method further comprises repeating the steps of:

receiving from the individual a selection of at least one scent additive from said scent additive choice,

adding to the fragrance the selected at least one scent additive to yield a fragrance blend customized for the individual, and

providing a sample of the fragrance blend to the individual for testing until the individual is satisfied.

In some embodiments, when the method is repeated, the second scent additive is the same as the scent additive used in the initial method. In some embodiments, the second scent additive is different.

In some embodiments, the sample is provided as a test strip, e.g. made of blotting paper. In some embodiments, the sample is provided in a tube. In some embodiments, the tube further contains a substrate such as ceramic wool or any other suitable absorbent material.

The compositions, methods, and uses described herein will be better understood by reference to the following exemplary embodiments and examples, which are included as an illustration of and not a limitation upon the scope of the invention.

### EXAMPLES

### Example 1

An individual selects Philosophy AMAZING GRACE eau de toilet as an initial fragrance. The individual is offered a scent additive choice chosen from bergamot oil, jasmine extract, and lavender oil. The individual selects bergamot oil. The selected scent additive is dispensed into a bottle. The fragrance is then dispensed into the bottle which is provided to the individual.

It is understood that the foregoing detailed description and accompanying examples are merely illustrative and are not to be taken as limitations upon the scope of the invention, which is defined solely by the appended claims and their equivalents. Various changes and modifications to the disclosed embodiments will be apparent to those skilled in the art. Such changes and modifications may be made without departing from the spirit and scope thereof.

All references (patent and non-patent) cited above are incorporated by reference into this patent application. The discussion of those references is intended merely to summarize the assertions made by their authors. No admission is made that any reference (or a portion of any reference) is relevant prior art (or prior art at all). Applicant reserves the right to challenge the accuracy and pertinence of the cited references.

1. A method for providing a fragrance blend customized for an individual, the method comprising:
providing a fragrance wherein the fragrance comprises perfuming ingredients belonging to all levels of a fragrance construct;
presenting the individual with a scent additive choice comprised of a plurality of scent additives;
receiving from the individual a selection of at least one scent additive from said scent additive choice, and
adding to the fragrance the selected at least one scent additive to yield a fragrance blend customized for the individual,
wherein the fragrance has a first fragrance, the selected scent additive has a second fragrance, and the fragrance blend has a third fragrance.

2. A method of enhancing a scent in a fragrance, the method comprising:
providing a fragrance wherein the fragrance comprises perfuming ingredients belonging to all levels of a fragrance construct;
presenting the individual with a scent additive choice comprised of a plurality of scent additives wherein the plurality of scent additives have been selected to enhance an olfactive note in the fragrance;
receiving from the individual a selection of at least one scent additive from said scent additive choice, and
adding to the fragrance the selected at least one scent additive in an amount sufficient to enhance an olfactive note in the fragrance to yield a fragrance blend having an enhanced scent,
wherein the fragrance has a first fragrance, the selected scent additive has a second fragrance, and the fragrance blend has a third fragrance.

3. The method of claim 1, wherein the fragrance is an existing commercial product.

4. The method of claim 1, wherein the fragrance is a classic product.

5. The method of claim 1, wherein the plurality of scent additives comprises musk oil, civet, castoreum, ambergris, plant perfumes, sandalwood oil, neroli oil, bergamot oil, lemon oil, lavender oil, sage oil, rosemary oil, peppermint oil, eucalyptus oil, menthol, camphor, verbena oil, citronella oil, coconut oil, salvia oil, clove oil, chamomile oil, costus oil, labdanum oil, broom extract, carrot seed extract, jasmine extract, mimosa extract, narcissus extract, olibanum extract, rose extract, acetophenonene and derivatives thereof, dimethylaniline derivatives, naphthaline derivatives, allyl caprate, alpha-amylcinnamic aldehyde, anethole, anisaldehyde, benzyl acetate, benzyl alcohol, benzyl propionate, borneol, cinnamyl acetate, cinnamyl alcohol, citral, citronellal, cumin aldehyde, cyclamen aldehyde, decanol, ethyl butyrate, ethyl caprate, ethyl cinnamate, ethyl vanillin, eugenol, geraniol, exenol, alpha-hexylcinnamic aldehyde, hydroxycitrolnellal, indole, iso-amyl acetate, iso amyl isovalerate, iso-eugenol, linalol, linalyl acetate, p-methylacetophenone, methyl anthranilate, methyl dihydrojasmonate, methyl eugenol, methyl-beta-naphthol ketone, methyl phenyl carbinyl acetate, musk ketol, musk xylol, 2,5,6-nonadienal, gamma-nonalactone, phenyl acetaldehyde dimethyl acetal, beta-phenylethyl alcohol, 3,3,5-trimethylcyclohexanol, gamma-undecalactone, undecenal, vanillin, metofluthrin, limonenem, terpinene gamma, calorie, helional, and mixtures thereof.

6. (canceled)

7. (canceled)

8. The method of claim 1, wherein the plurality of scent additives comprises one or more perfuming ingredients having contrasting notes to one or more olfactive notes of the fragrance.

9. The method of claim 1, wherein the plurality of scent additives comprises one or more perfuming ingredients having one or more olfactive notes of the fragrance.

10. (canceled)

11. The method of claim 1, wherein the selected scent additive is a perfuming ingredient having a contrasting note to one or more olfactive notes of the fragrance.

12. The method of claim 1, wherein the selected scent additive is a perfuming ingredient having one or more olfactive notes of the fragrance.

13. (canceled)

14. (canceled)

15. (canceled)

16. The method of claim 1, wherein the fragrance blend is formulated as a perfume, perfume water, Eau de toilette, Eau de cologne, Eau de parfum, fragrance mist, or air freshener.

17. (canceled)

18. (canceled)

19. The method of claim 1, further comprising:
presenting the individual with an effect additive choice comprised of a plurality of effect additives;
receiving from the individual a selection of an effect additive from said plurality of effect additives, and
adding to the fragrance the selected effect additive.

20. The method of claim 19, wherein the effect additive choice comprises particles that part a tactile sensory experience to an end user.

21. The method of claim 19, wherein the effect additive choice comprises additives that provide a visual effect.

22. (canceled)

23. (canceled)

24. (canceled)

25. The method of claim 1, wherein after the selected at least one scent additive is added to the fragrance to yield a fragrance blend, the method further comprises providing a sample of the fragrance blend to the individual for testing.

26. The method of claim 25, wherein the individual is satisfied with the sample, and the method further comprises providing the fragrance blend to the individual.

27. The method of claim 25, wherein the individual is not satisfied with the sample, and the method further comprises repeating the steps of:
receiving from the individual a selection of at least one scent additive from said scent additive choice,
adding to the fragrance the selected at least one scent additive to yield a fragrance blend customized for the individual, and
providing a sample of the fragrance blend to the individual for testing until the individual is satisfied.

28. A fragrance blend prepared by the method of claim 1.

29. A fragrance blend comprising a fragrance and a scent additive.

30. (canceled)

31. The fragrance blend of claim 28, wherein the scent additive is present in an amount sufficient to enhance an olfactive note in the fragrance.

32. The fragrance blend of claim 28, wherein the scent additive comprises a perfuming ingredient having a contrasting note to the one or more notes of the fragrance.

33. The fragrance blend of claim 28, wherein the scent additive comprises a perfuming ingredient having one or more notes of the fragrance.

34. The fragrance blend of claim 28, wherein the fragrance blend is formulated as a perfume, perfume water, Eau de toilette, Eau de cologne, Eau de parfum, fragrance mist, or air freshener.
